(19) United States
(12) Patent Application Publication
(10) Pub. No.: US 2026/0083956 A1
(43) Pub. Date: Mar. 26, 2026

(54) MECHANICAL CIRCULATORY SUPPORT SYSTEM WITH REPOSITIONING SHEATH

(71) Applicant: BOSTON SCIENTIFIC SCIMED, INC., Maple Grove, MN (US)

(72) Inventors: Victor Kotov, St. Louis Park, MN (US); Foss Jiao, Woodbridge (CA); Pooja Mani, Etobicoke (CA); Benjamin Michael Nitti, Stillwater, MN (US)

(73) Assignee: BOSTON SCIENTIFIC SCIMED, INC., Maple Grove, MN (US)

(21) Appl. No.: 19/333,716

(22) Filed: Sep. 19, 2025

Related U.S. Application Data
(60) Provisional application No. 63/697,161, filed on Sep. 20, 2024.

(51) Int. Cl. A61M 60/174; A61M 60/508; A61M 60/865

(52) U.S. Cl. CPC A61M 60/174; A61M 60/508; A61M 60/865

(57) ABSTRACT

A medical device system may include a medical device, an elongate shaft, a first sheath, and a second sheath. The elongate shaft may couple with the medical device and extend proximally from the medical device. The first sheath may have an elongate tube configured to expand from a first inner diameter to a second inner diameter. The first sheath and the second sheath may longitudinally advance over the elongate shaft and a distal end of the second sheath may advance through the lumen and extend distally beyond a distal end of the first elongate tube into a vascular access site. The system may include a closure device configured to close the vascular access site around the second sheath. The medical device may be or include a blood pump configured to pump blood from a ventricle of a heart of a patient to vasculature of the patient.

### CROSS REFERENCE TO RELATED APPLICATIONS

The present application claims the benefit of US Provisional Patent Application Serial No. 63/697,161, filed September 20, 2024, the disclosure of which is incorporated herein by reference.

### TECHNICAL FIELD

The present disclosure pertains to a sheath configuration for a mechanical circulatory support system. More particularly, the present disclosure pertains to a sheath system of a mechanical circulatory support system and/or other medical device systems for use at a vascular access site.

### BACKGROUND

In various procedures for delivering intravascular medical devices, an introducer sheath is inserted into a blood vessel of a patient, for example a femoral artery, and one or more medical devices may be advanced through the sheath and into the patient’s vasculature. In various instances, the medical devices include catheters or other devices, such as a blood pump. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including percutaneous blood pumps and/or other medical devices and associated devices.

A first example may be a medical device system including a medical device, an elongate shaft coupled with the medical device and extending proximally from the medical device, a first sheath having a first hub, a first elongate tube extending in a distal direction from the first hub, and a first length extending from a proximal end of the first hub to a distal end of the first elongate tube, the first elongate tube has a lumen with an inner diameter and the inner diameter is expandable from a first inner diameter to a second inner diameter, and a second sheath having a second elongate tube, the second elongate tube having a tube length that is longer than the sheath length, and wherein the first sheath and the second sheath may be configured to longitudinally advance over the elongate shaft and a distal end of the second sheath may be configured to advance through the lumen and extend distally beyond the distal end of the first elongate tube.

Alternatively or additionally to any of the examples above, the second sheath may comprise a second hub and the second elongate tube may extend in the distal direction from a distal end of the second hub.

Alternatively or additionally to any of the examples above, the first sheath may be an introducer sheath and the first elongate tube may be configured to expand to the second inner diameter in response to receiving the medical device in the lumen.

Alternatively or additionally to any of the examples above, the first inner diameter may be in a range of 2.33 millimeters (mm) to 3 mm and the second inner diameter may be in a range of 4.67 mm to 6.67 mm.

Alternatively or additionally to any of the examples above, the second sheath may be a single lumen sheath.

Alternatively or additionally to any of the examples above, the system may further include a closure device configured to extend around the second elongate tube and close a patient access site within which the second elongate tube is extending.

Alternatively or additionally to any of the examples above, the closure device may be a suture.

Alternatively or additionally to any of the examples above, the first sheath may be separable into two or more portions for removal from the elongate shaft after the blood pump has been inserted through the lumen.

Alternatively or additionally to any of the examples above, the system may further include a housing, wherein the elongate shaft may extend between a proximal end of the medical device and a distal end of the housing and the second sheath may be located about the elongate shaft between the housing and the medical device.

Alternatively or additionally to any of the examples above, the medical device may be a blood pump configured to pump blood from a ventricle of a heart of a patient to vasculature of the patient.

In another example, a method of using a medical device system may include positioning a closure device at a vascular access site of a patient, advancing a medical device and an elongate shaft extending proximally from the medical device pump through a first sheath extending into the vascular access site, advancing a distal end of a second sheath over the elongate shaft and through a distal end of the first sheath to a location distal of the distal end of the first sheath, and closing the vascular access site around the second sheath using the closure device.

Alternatively or additionally to any of the examples above, the closure device may be a suture, positioning the closure device at the vascular access site may include threading the suture through skin of the patient, and closing the vascular access site around the second sheath may include tying the suture around the second sheath to close the vascular access site around the second sheath.

Alternatively or additionally to any of the examples above, the second sheath may include a taper and advancing the distal end of the second sheath to a location distal of the distal end of the first sheath may include positioning the taper to control hemostasis at the vascular access site.

Alternatively or additionally to any of the examples above, the method may further include advancing the first sheath through the vascular access site after positioning the closure device at the vascular access site.

Alternatively or additionally to any of the examples above, the method may further include removing the first sheath from the vascular access site and removing the first sheath from the second sheath and the elongate shaft.

Alternatively or additionally to any of the examples above, the first sheath may include an elongate tube with an inner diameter configured to expand from a first inner diameter to a second inner diameter as the medical device is advanced through the first sheath.

In a further example, a medical device system may include a first sheath having a first hub and a first elongate tube extending in a distal direction from the first hub, the first elongate tube has a lumen with an inner diameter, a medical device, a housing, an elongate shaft coupled with the medical device and extending proximally from the medical device to the housing, a second sheath having a second elongate tube positioned about the elongate shaft, the second elongate tube has an outer diameter that is less than the inner diameter of the first elongate tube, and a closure device configured to close a vascular access site around the second sheath.

Alternatively or additionally to any of the examples above, the second sheath may be configured to adjust between a first position entirely proximal of the first sheath and a second position in which the second elongate tube extends distal of a distal end of the first sheath.

Alternatively or additionally to any of the examples above, the first elongate tube may be configured to expand from a first inner diameter to a second inner diameter.

Alternatively or additionally to any of the examples above, the closure device may be a suture.

The above summary of some embodiments is not intended to describe each disclosed embodiment or every implementation of the present disclosure. The Figures and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative configuration of a mechanical circulatory support (MCS) system including a percutaneous blood pump;

FIG. 2 is a schematic partial cross-section view of a portion of an illustrative configuration of an MCS system including a percutaneous blood pump;

FIG. 3A is a schematic partial cross-section view of a portion of an illustrative configuration of an MCS system including a percutaneous blood pump, in a first configuration;

FIG. 3B is a schematic partial cross-section view of the portion of the illustrative configuration of the MCS system depicted in FIG. 3A, in a second configuration;

FIG. 4 is a schematic partial cross-section view of a portion of an illustrative configuration of an MCS system including a closure device; and

FIGS. 5A-5G depict a schematic flow of an illustrative technique for using an MCS system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

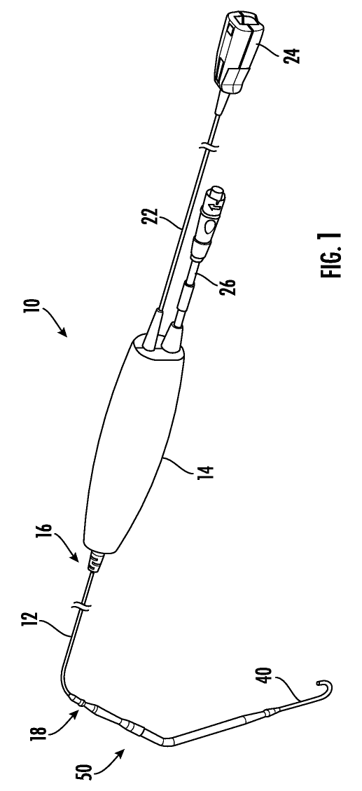

FIG. 1 illustrates a perspective view of a mechanical circulatory support (MCS) system 10 including a percutaneous blood pump 50 located at a distal end region thereof. The blood pump 50 may be configured to pump blood from a ventricle of a heart of a subject (e.g., a patient) to vasculature of the subject and/or between other portions of the circulatory system.

The MCS system 10 may be coupled to or include the blood pump 50, with an elongate shaft 12 extending proximally from the percutaneous blood pump 50 and a distal tip 40 extending distally from the blood pump 50. The elongate shaft 12 may be an elongate tube with one or more elongate members, such as wires, extending therein. A proximal end 16 of the elongate shaft 12 may be coupled to a housing 14 (e.g., a housing of a handle, of a control module, etc.) and a distal end 18 of the elongate shaft 12 may be coupled to the percutaneous blood pump 50, such that the elongate shaft 12 may extend between the housing 14 and the blood pump 50. An electrical cable 22 may extend from the housing 14 to a connector 24 at a proximal end thereof. The connector 24 may be configured to be connected to a controller (not shown) for controlling the blood pump 50, such as providing electrical power, control signals, etc. to the blood pump 50. The MCS system 10 may also include an extension 26 connectable to the controller for sending and/or receiving signals, such as from one or more sensors during operation of the blood pump 50. The MCS system 10 may included additional and/or alternative components and/or arrangements of components.

The elongate shaft 12 (e.g., an elongate tube of the elongate shaft 12) may have any suitable outer diameter. For example, the elongate shaft 12 may have an outer diameter in a range 2 mm (6 French (Fr)) to 4 mm (12 Fr) and/or other suitable outer diameters. In one example, the elongate shaft 12 may have an outer diameter of 2.33 mm (7 Fr), but other suitable outer diameters are contemplated

When placing the blood pump 50 in a subject (e.g., a patient), the blood pump 50 may be inserted through an introducer sheath inserted into a vascular access site of the subject. In some cases, the introducer sheath may be a large bore sheath configured to receive and facilitate the passage therethrough of the blood pump 50 and the elongate shaft 12. In some cases, such an introducer sheath may have an outer diameter that tends to block the free flow of blood through an artery or one or more other vessels of the subject in which the introducer sheath is located. For example, the introducer sheath may block the free flow of blood through a femoral artery, an axillary artery, a subclavian artery, etc. to an extremity distal thereof. In one example, the introducer sheath may block the free flow of blood through the femoral artery to a leg of the subject.

The introducer sheath may be expandable or non-expandable. To facilitate perfusion of blood to the leg or other location of the subject, the introducer sheath may be an expandable introducer sheath. For example, the introducer sheath may have an elongate shaft or tube formed from an elastomeric or other resilient material having a first inner diameter and/or first outer diameter when in a first state (e.g., an original state) and a second inner diameter and/or second outer diameter greater than the first inner diameter and/or the first outer diameter, respectively, when in a second state (e.g., an expanded state, such as when a large bore medical (e.g., the blood pump 50) is being passed therethrough). When an expandable sheath is utilized, the smaller first outer diameter may facilitate blood perfusion through an artery or other vessel in which the introducer sheath is inserted prior to when the medical device is inserted within a portion of the introducer sheath in the artery or other vessel, while still allowing passage of medical devices by being able to expand to the second inner diameter when inserting the medical device into the artery or other vessel.

Further, to facilitate perfusion of blood to the leg or other portion of the subject after the blood pump 50 or other suitable medical device is delivered into the vessel and/or to a target site, the introducer sheath may be swapped out for a smaller outer diameter sheath, such as a repositioning sheath. To facilitate removal of the introducer sheath from around the elongate shaft 12 and/or the repositioning sheath after inserting the repositioning sheath through the introducer sheath and the vascular access site, the introducer sheath may be a separable and/or peel-away sheath, where the introducer sheath may be configured to split along a perforated line, a pull line, etc. extending along an entirety of or at least part of a length of the introducer sheath, and/or may be removable from the elongate shaft 12 and/or repositioning sheath in one or more other suitable manners. Alternatively or additionally, the introducer sheath may be removed from the vascular access site and maintained over the repositioning sheath and/or the elongate shaft 12 after inserting the repositioning sheath into and/or through the vascular access site.

When an expandable introducer sheath is utilized and/or a repositioning sheath having an outer diameter less than an outer diameter of the blood pump 50 are utilized, a gap may form at the vascular access site between a circumference of an opening at the vascular access site and the expandable introducer sheath and/or the repositioning sheath through which blood may exit the body of the patient. To prevent or mitigate such external bleeding, one or more closure devices may be utilized to close the vascular access site around the expandable introducer sheath and/or the repositioning sheath.

The concepts discussed herein may include utilizing an introducer sheath and a repositioning sheath that are both configured to receive and advance over the elongate shaft 12. In some configurations, the repositioning sheath may have a length that is longer than a length of the introducer sheath such that a distal end of the repositioning sheath may be inserted into an access site to a location distal of a distal end of the introducer sheath before, during, or after the introducer sheath is fully withdrawn from the access site, while a proximal hub of the repositioning sheath is proximal of a proximal hub of the introducer sheath. Further, a closure device (e.g., a suture and/or other suitable closure device) may be utilized to close the access site around the repositioning sheath.

The concepts discussed herein describe utilization of an introducer sheath and a repositioning sheath for delivery of MCS systems as an example. Other suitable medical devices can utilize and would benefit with use of the discussed introducer and repositioning sheath for deployment through a vessel and/or to a target site. Illustrative medical devices include, but are not limited to, structural heart valves, percutaneous coronary intervention guidance systems, drug eluting and non-drug eluting stents, drug eluting and non-drug eluting balloons, guide or diagnostic catheters, embolic protection filters, thrombectomy systems, atherectomy systems, and denervation or ablation systems.

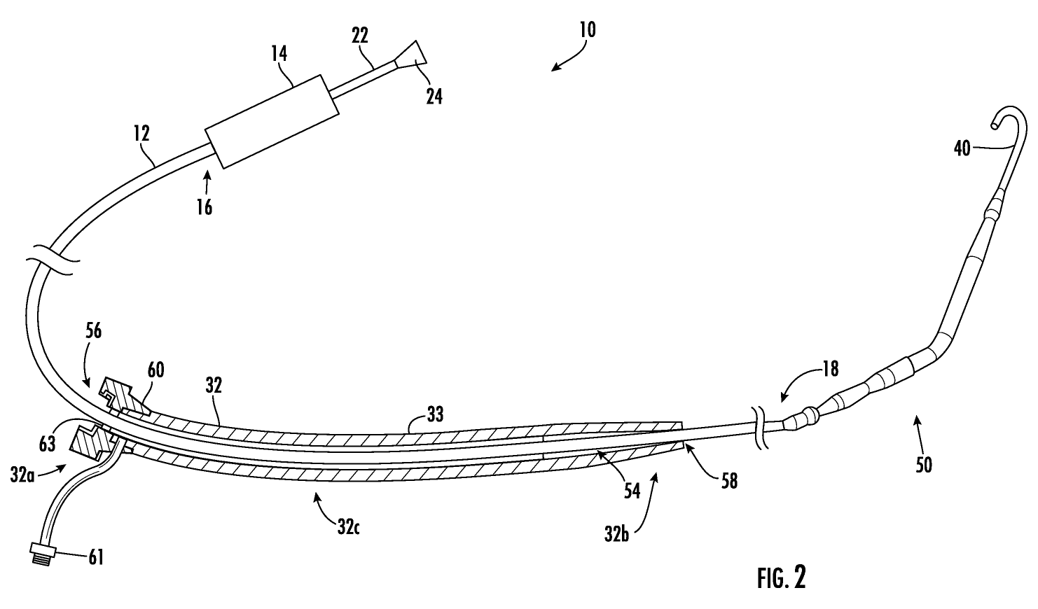

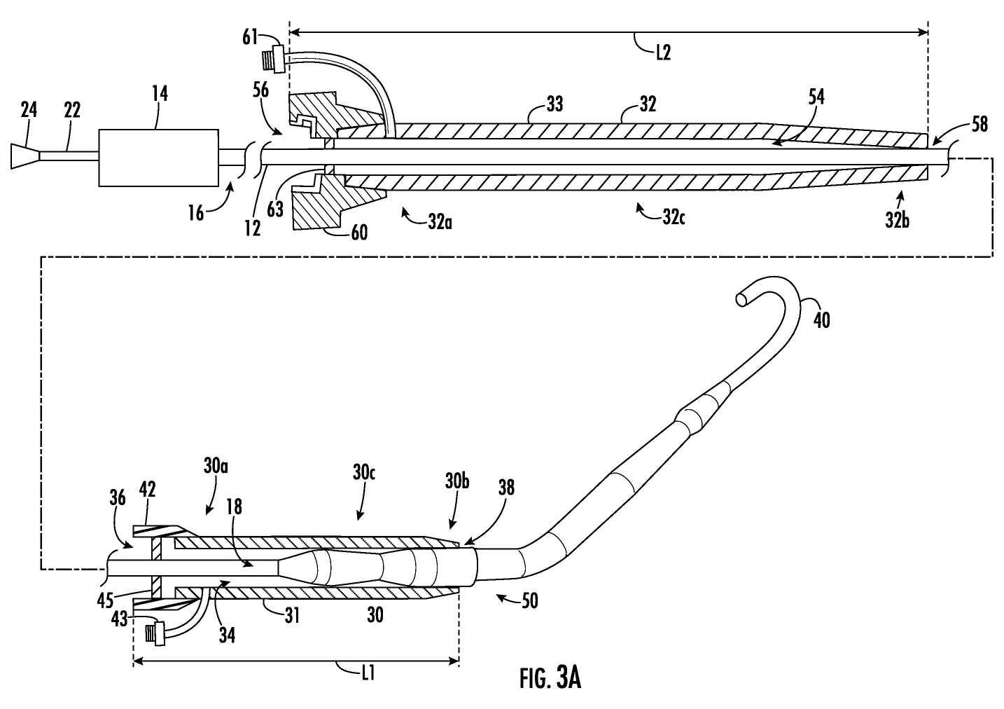

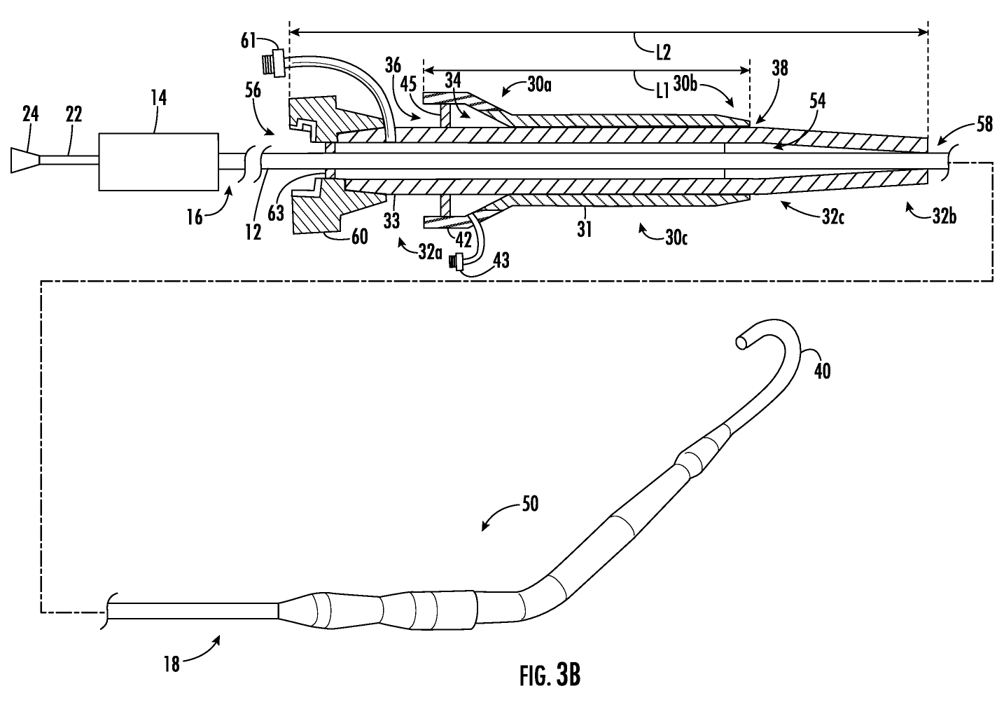

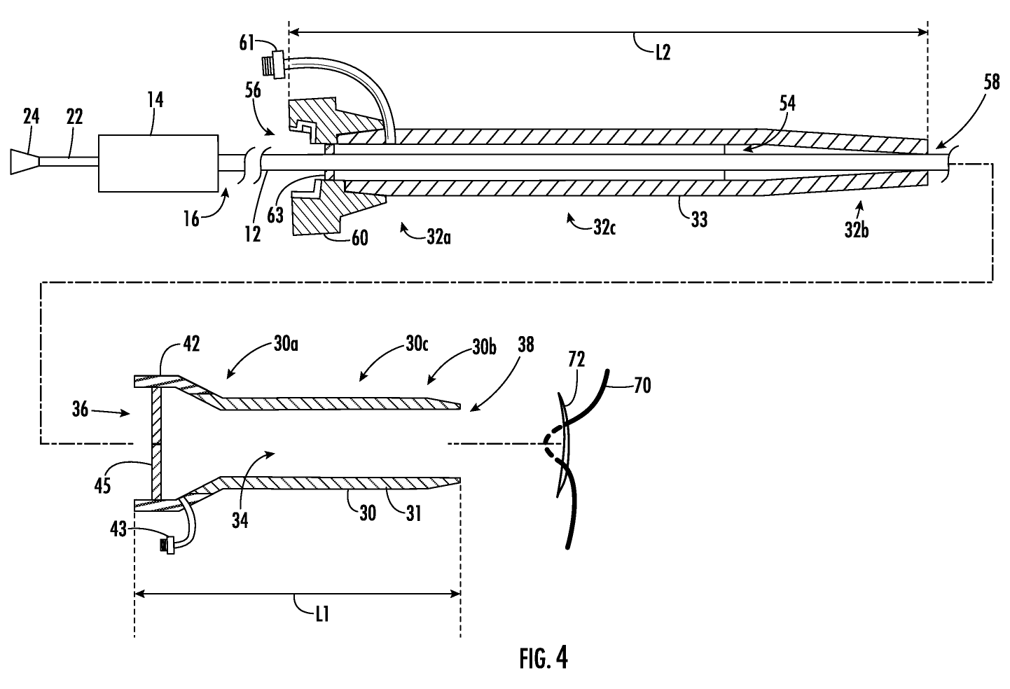

FIG. 2 depicts a schematic partial cross-section view of an illustrative configuration of a portion of the MCS system 10. The portion of the MCS system 10 depicted in FIG. 2 may include, among other components, the elongate shaft 12 extending distally from the housing 14 to the blood pump 50 and a repositioning sheath 32 (e.g., a second sheath) positioned about or along the elongate shaft 12 between the housing 14 and the blood pump 50, where the repositioning sheath 32 is depicted in cross-section. The MCS system 10 may include additional and/or alternative components including, but not limited to, an introducer sheath (e.g., a first sheath, discussed in greater detail with respect to FIGS. 3A and 3B) and a closure device (e.g., discussed in greater detail with respect to FIG. 4).

Each of the elongate shaft 12, the repositioning sheath 32 and/or other components of the MCS system 10 may be configured in any suitable manner with respect to one or more other components of the MCS system 10. In some example configurations of the MCS system 10, the elongate shaft 12 and the repositioning sheath 32 may be configured to rotate and/or translate longitudinally relative to one another. In some examples, the repositioning sheath 32 may be co-axial with the elongate shaft 12. Other suitable configurations of the repositioning sheath 32 relative to the elongate shaft 12 are contemplated.

The repositioning sheath 32 may have any suitable configuration. In some example configurations, the repositioning sheath 32 may have a fixed curve when in a resting position, but other suitable configurations are contemplated. Further, the repositioning sheath 32 may include or define any suitable number of lumens. Although FIG. 2 depicts only a single lumen 54 of the repositioning sheath 32, the repositioning sheath 32 may have a single lumen, two lumens, three lumens, and/or other suitable number of lumens as desired. In one example, the repositioning sheath 32 may include an elongate tube 33 (e.g., a second elongate tube) extending in a distal direction from a hub 60 with a port 61, where the elongate tube 33 may entirely or at least partially define the lumen 54 and/or other lumens.

The repositioning sheath 32 may include a proximal end region 32a that includes the hub 60 and a distal end region 32b. A body region 32c of the repositioning sheath 32 may extend between the proximal end region 32a and the distal end region 32b, where the elongate tube 33 of the repositioning sheath 32 may define, entirely or at least in part, the body portion 32c. One or more of the proximal end region 32a, the distal end region 32b, and the body region 32c may define the lumen 54 and/or other lumens of the repositioning sheath 32. Further, the repositioning sheath 32 may include a proximal opening 56 and a distal opening 58 with the lumen 54 extending between the proximal opening 56 and the distal opening 58.

An outer diameter of the repositioning sheath 32 may have any suitable configuration between the proximal end region 32a and the distal end region 32b. In some examples, the outer diameter of the repositioning sheath 32 may change along a length of the repositioning sheath 32. In one example, the distal end region 32b of the repositioning sheath 32 may have a smaller outer diameter than the proximal end region 32a and/or the body region 32c, where the outer diameter of the repositioning sheath 32 may taper up in a proximal direction between or at the distal end region 32b and the body region 32c. The smaller outer diameter at the distal end region 32b may facilitate mitigating ischemia due to a relatively small outer diameter portion of the repositioning sheath 32 being located in the vessel of the subject, may facilitate hemostasis control at an access site, and/or may facilitate reinserting the repositioning sheath 32 through the access site and into the subject if needed. Alternatively, the tapering of the outer diameter of the repositioning sheath 32 may be omitted and the repositioning sheath 32 may have a constant outer diameter along a length of the elongate tube 33.

The repositioning sheath 32 may be formed from one or more suitable materials. Example suitable materials include, but are not limited to, polymeric and/or metallic materials. In one example configuration, the elongate tube 33 of the repositioning sheath 32 may be formed from a flexible polymer and the hub 60 may be formed from a rigid polymer, but other suitable configurations are contemplated. In some instances, the repositioning sheath 32 may include one or more additional surface coatings on the interior surface defining the lumen 54 and/or the exterior surface, which may include, but are not limited to, silicone, polyethylene terephthalate (PET), and/or other suitable coating materials.

The repositioning sheath 32 may have any suitable inner diameter, which defines the diameter of the lumen 54, and any suitable outer diameter. In some examples, the inner diameter of the elongate tube 33 of the repositioning sheath 32 may be in a range of 1.67 mm (5 Fr) to 4.33 mm (13 Fr). In some examples, the outer diameter of the elongate tube 33 of repositioning sheath 32 may be in a range of 2 mm (6 Fr) to 4.67 mm (14 Fr). In one example, the outer diameter of the elongate tube 33 of repositioning sheath 32 may be 2.67 mm (8 Fr), but other suitable diameters are contemplated. In some examples, the repositioning sheath 32 may have any suitable outer diameter that is smaller than the outer diameter of the introducer sheath 30 so as to better allow blood perfusion through the blood vessel of the subject in which the elongate shaft 12 is inserted and reduce a maintained diameter opening at the access site of the subject while the blood pump 50 is positioned at a target site (e.g., during a dwell time). In some examples, the outer diameter of the repositioning sheath 32 may be less than an inner diameter of the introducer sheath 30 to facilitate the repositioning sheath 32 advancing (e.g., in a proximal and/or distal axial direction) through the introducer sheath 30, but this is not required and the introducer sheath 30 may be configured to stretch or flex radially outward to allow the repositioning sheath 32 to pass through the introducer sheath 30.

The hub 60 may be at and/or coupled with a proximal end of the elongate tube 33 and may be considered part of the repositioning sheath 32, but this is not required. The hub 60 may define, at least in part, the proximal opening 56 of the repositioning sheath 32 and may facilitate receiving and/or coupling with one or more components of the MCS system 10.

The hub 60 may include one or more coupling mechanisms configured to couple with the elongate shaft 12 (e.g., an elongate shaft lock) to facilitate releasably fixing the elongate shaft 12 relative to the repositioning sheath 32. Example suitable coupling mechanisms include, but are not limited to, a clip, a clasp, a friction fit connection, a rotational tightening mechanism, threads, a valve, a seal, and/or other suitable type of coupling mechanism. In some examples, the hub 60 may include a valve or seal 63 configured to couple with the elongate shaft 12 and/or create hemostasis at the hub 60.

The valve or seal 63, when included, may be any suitable type of valve or seal configured to block blood flow through, for example, the lumen 54. In some examples, the valve or seal 63 may be formed from a silicone disc with a cross-cut configured to receive the elongate shaft 12 and/or other suitable components received within the lumen 54, and allow the repositioning sheath 32 to pass over and seal around the elongate shaft 12 and/or other components. However, it is contemplated that the valve or seal 63 may be formed from one or more additional or alternative materials and/or include one or more other suitable cuts or openings configured to receive and seal around the elongate shaft 12 and/or other components received within the lumen 54.

FIGS. 3A and 3B depict a schematic partial cross-sectional view of an illustrative configuration of a portion of the MCS system 10. The portion of the MCS system 10 depicted in FIGS. 3A and 3B may include the housing 14, the blood pump 50, the elongate shaft 12 extending distally from the housing 14 to the blood pump 50, an introducer sheath 30 (e.g., a first sheath), and the repositioning sheath 32 (e.g., a second sheath), among other suitable components and/or features.

In some cases, the introducer sheath 30 may be used for facilitating passage of various medical devices, such as the elongate shaft 12, the blood pump 50, etc., through an access site of the subject and into a blood vessel of the subject. The introducer sheath 30 may include a proximal end region 30a and a distal end region 30b. A body region 30c of the introducer sheath 30 may extend between the proximal end region 30a and the distal end region 30b, where one or more of the proximal end region 30a, the distal end region 30b, and the body region 30c may define one or more lumens 34 of the introducer sheath 30. Further, the introducer sheath 30 may include a proximal opening 36 and a distal opening 38 with the lumen 34 extending between the proximal opening 36 and the distal opening 38. The lumen 34 may have and/or define an inner diameter of the introducer sheath 30.

The introducer sheath 30 may have any suitable diameter defined by an inner surface of the introducer sheath 30. In some examples, the introducer sheath 30 (e.g., configured as a splittable or cuttable introducer sheath) may have a fixed, constant inner diameter along its length. In some examples, the introducer sheath 30 may have an adjustable or expandable inner diameter between a minimum diameter and a maximum diameter. The introducer sheath 30 may have an inner diameter in a range of 2 millimeters (mm) (6 Fr) to 7 mm (21 Fr). In one example configuration in which the introducer sheath 30 may have a fixed, constant outer diameter along the length of the introducer sheath 30, the introducer sheath 30 may have a diameter in a range of about 4.67 mm (14 Fr) to about 6.67 mm (20 Fr). In one example configuration in which the introducer sheath 30 may be expandable, the introducer sheath 30 may include an elongate tube 31 extending in a distal direction from a hub 42 and configured to expand, where the elongate tube 31 may define the outer diameter of the introducer sheath 30 when in an original condition (e.g., a first inner diameter) within a range of 2.33 mm (7 Fr) to 3 mm (9 Fr) and the outer diameter of the introducer sheath 30 when in an expanded condition (e.g., a second inner diameter) in a range of 4.67 mm (14 Fr) to 6.67 mm (20 Fr). In one example configuration of introducer sheath 30 configured to expand from an original configuration to an expanded configuration, the introducer sheath 30 may have a 2.67 mm (8 Fr) outer diameter when in the original configuration and may have 5.33 mm (16 Fr) outer diameter when in a maximum expanded configuration, but other suitable configurations are contemplated. A diameter of the lumen 34 or an inner diameter of the introducer sheath 30 defining the lumen 34 may be configured to adjust with the outer diameter of the introducer sheath 30 to facilitate receiving medical devices therethrough. When the introducer sheath 30 is an expandable introducer sheath, the introducer sheath 30 may remain in its expanded configuration after the inner diameter and/or the outer diameter have been expanded, but other suitable configurations are contemplated.

The introducer sheath 30 may be formed from one or more suitable materials. Example suitable materials include, but are not limited to, polymeric, elastomeric, and/or metallic materials. In some instances, the introducer sheath 30 may include one or more additional surface coatings on the interior surface defining the lumen 34 and/or the exterior surface, which may include, but are not limited to, silicone, polyethylene terephthalate (PET), and/or other suitable coating materials. In one example, the hub of the introducer sheath 30 may be formed from a rigid polymer material and the elongate tube 31 of the introducer sheath 30 may be formed from an elastomer material configured to stretch and/or expand between a minimum diameter and a maximum diameter based, at least in part, on a diameter of component inserted into the lumen 34 of the introducer sheath 30.

As discussed, the introducer sheath 30 (e.g., at the proximal end region thereof) may include the hub 42 (e.g., a proximal hub, such as a hemostasis valve hub, and/or other suitable hub) and the elongate tube 31 extending in a distal direction from the hub 42 that defines, entirely or at least in part, the body region 30c of the introducer sheath 30. The hub 42 may include a port 43 and provide access to the lumen 34. The hub 42 may be configured for hemostasis by, for example, preventing or mitigating blood leakage out of the introducer sheath 30 during use via, for example, a valve or a seal 45 and/or other suitable hemostasis component. For example, a medical device, such as the elongate shaft 12 and/or the blood pump 50, may be inserted through the hub 42, the valve and/or seal 45, and the lumen 34 of the introducer sheath 30 and into the blood vessel of the subject, where the hub 42 may maintain hemostasis between the medical device, the introducer sheath 30, and the external surroundings.

The valve or seal 45 may be any suitable type of valve or seal configured to block blood flow and/or air or other gasses through, for example, the lumen 34. In some examples, the valve or seal 45 may be or and/or may be formed from a silicone disc with a cross-cut configured to receive the blood pump 50, the elongate shaft 12, the repositioning sheath 32, and/or other suitable components, and allow the introducer sheath 30 to pass over and seal around the components received within the lumen 34. However, it is contemplated that the valve or seal 45 may be formed from one or more additional or alternative materials and/or include one or more other suitable cuts or openings configured to receive and seal around the components received within the lumen 34.

In some examples, the hub 42 may be or may include a tightening port. When included, the tightening port may be any suitable type of tightening port. For example, the tightening port may engage the elongate shaft 12 in response to rotating a rotational actuator of the tightening port, adjusting a clip of the tightening port, and/or the tightening port may engage the elongate shaft 12 with one or more additional or alternative coupling techniques discussed herein (e.g., the valve or seal 45). Further, in some cases, the tightening port and/or other suitable portions of the hub 42 may be configured to receive and couple with the hub 60 of the repositioning sheath 32 and/or other portions of the repositioning sheath 32.

The introducer sheath 30 may have any suitable length L1 (e.g., a sheath length). In some examples, the length L1 of the introducer sheath 30 may extend from a proximal end of the introducer sheath 30 at or proximate the proximal opening 36 to a distal end of the introducer sheath 30 at or proximate the distal opening 38. Example suitable lengths of the introducer sheath 30 include, but are not limited to, lengths in a range of about 13 centimeters (cm) to about 35 cm. In one example, the introducer sheath 30 may have a length L1 of 12 cm, but other suitable lengths for the introducer sheath 30 are contemplated.

The introducer sheath 30 may be configured to remain extended over the repositioning sheath 32 after being removed from the access site and/or may be configured to be removed from extending around the repositioning sheath 32 and/or the elongate shaft 12. In some examples, the introducer sheath 30 may be separable into two or more portions for removal from along the elongate shaft 12 and/or the repositioning sheath 32 after the blood pump 50 has been inserted through the introducer sheath 30 (e.g., the lumen 34 of the introducer sheath 30) and into the vasculature of the patient and/or positioned at a target site. In some examples, the introducer sheath 30 may include a perforation line, a pull line, and/or other separation inducing feature such that the introducer sheath 30, including the hub 42, is induced to split into two or more components in response to radially outward forces acting on the introducer sheath. Such a separable introducer sheath 30 may be a peel apart or peel away sheath. In some examples, the introducer sheath 30 may be configured to be cut to split apart and remove the introducer sheath 30 from the repositioning sheath 32. Other suitable configurations of a separable introducer sheath 30 are contemplated.

The repositioning sheath 32 may be configured to replace the introducer sheath 30 at the access site to reduce an outer diameter of a sheath extending into the vessel of the subject after the blood pump 50 and/or other medical device passes through the introducer sheath 30. The repositioning sheath 32 may be configured to advance over the elongate shaft 12 and through the lumen 34 of the introducer sheath 30 to the access site of the subject. For example, once the blood pump 50 has been positioned in the blood vessel of the subject and/or at a target site within the subject, the repositioning sheath 32 (e.g., the distal end region 32b of the repositioning sheath 32) may be advanced toward the access site through the introducer sheath 30 to a location distal of a distal end of the introducer sheath 30 and positioned across the access site and in the blood vessel of the subject. The repositioning sheath 32 may be inserted into the access site and the blood vessel of the subject before, during, and/or after the introducer sheath 30 has been removed from the access site.

When positioned in the blood vessel of the subject, the repositioning sheath 32 may maintain access to the vasculature (e.g., arterial vasculature) of the patient and facilitate repositioning of the blood pump 50 at or proximate the target site within the subject manipulating the elongate shaft 12. The repositioning sheath 32 may facilitate increased blood perfusion through the blood vessel relative to the blood perfusion that occurs when the introducer sheath 30 is in the blood vessel due to the repositioning sheath 32 having a smaller outer diameter than the introducer sheath 30. Further, the repositioning sheath 32 may be less stiff or rigid than the introducer sheath 30, which may reduce trauma to the subject at the access site and mitigate effects that movements of the MCS system 10 proximal of the access site have on a position of the blood pump 50 at the target site.

The elongate tube 33 of the repositioning sheath 32 may have any suitable length L2 (e.g., a tube length). In some examples, the length L2 of the elongate tube 33 may extend from a distal end of the hub 60 to a distal end of the repositioning sheath 32 at or proximate the distal opening 58. Example suitable lengths of the elongate tube 33 include, but are not limited to, lengths in a range of about 10 cm to about 40 cm. In one example configuration, the elongate tube 33 may have a length of 32 cm, but other suitable lengths are contemplated. The length L2 of the elongate tube 33 may be configured to be longer than the length L1 of the introducer sheath 30, such that a distal end of the repositioning sheath 32 is configured to advance through the access site into a blood vessel distal of the distal end of the introducer sheath 30, while allowing the introducer sheath 30 to be advanced proximally (e.g., withdrawn) from the access site over the repositioning sheath 32 such that the hub 60 of the repositioning sheath 32 may couple with or be positioned proximate the hub 42 of the introducer sheath 30 and the distal end of the introducer sheath 30 is proximal of the access site. In some examples, when the introducer sheath 30 is configured as a separable introducer sheath, the elongate tube 33 may have the length L2 that is configured to be similar to the length L1 of the introducer sheath 30, but longer than the length L1 of the introducer sheath 30. Other suitable lengths L2 of the elongate tube 33 are contemplated.

The repositioning sheath 32 may have any suitable outer diameter, as discussed, that is smaller than the outer diameter of the introducer sheath 30 so as to better allow blood perfusion through the blood vessel of the subject in which the elongate shaft 12 is inserted and reduce a maintained diameter opening at the access site of the subject while the blood pump 50 is positioned at a target site (e.g., during a dwell time). In some examples, the outer diameter of the repositioning sheath 32 may be less than an inner diameter of the introducer sheath 30 to facilitate passing the repositioning sheath 32 through the introducer sheath 30, but this is not required and the introducer sheath 30 may be configured to stretch or flex radially outward to allow the repositioning sheath 32 to pass through the introducer sheath 30.

FIG. 3A schematically depicts the blood pump 50 being inserted through an expandable configuration of the introducer sheath 30. As shown in FIG. 3A, as the blood pump 50 is advanced through the lumen 34 of the introducer sheath 30, the introducer sheath 30 expands (e.g., the inner diameter and the outer diameter of the introducer sheath 30 expand) such that the inner diameter is sufficient to allow the blood pump 50 to pass through the introducer sheath 30. The repositioning sheath 32 may be entirely proximal of the introducer sheath 30 (e.g., in a first position) as the blood pump 50 is inserted through the introducer sheath 30, but other suitable configurations are contemplated.

FIG. 3B schematically depicts the blood pump 50 inserted through the introducer sheath 30 and the repositioning sheath 32 inserted through the lumen 34 of the introducer sheath 30. The lumen 34 of the introducer sheath 30 may be sized to receive the repositioning sheath 32 without expanding, but other suitable configurations are contemplated. Further, as discussed, the length L2 of the elongate tube 33 of the repositioning sheath 32 may be longer than the length L1 of the introducer sheath 30 such that the introducer sheath 30 may be withdrawn from the access site over the repositioning sheath 32 while the repositioning sheath 32 is inserted through the vascular access site and into the vasculature of the patient, such that the repositioning sheath 32 may extend distal of a distal end of the introducer sheath 30 (e.g., a second position). In some examples, the elongate tube 33 of the repositioning sheath 32 may have a length that is longer than the elongate tube 31 of the introducer sheath 30, but other suitable configurations are contemplated. Although not required, the hub 60 of the repositioning sheath 32 may be configured to be secured to the hub 42 of the introducer sheath 30.

FIG. 4 depicts a schematic partial cross-section, exploded view of an illustrative configuration of a portion of the MCS system 10. The portion of the MCS system 10 depicted in FIG. 4 may include the housing 14, the elongate shaft 12 extending distally from the housing 14, the introducer sheath 30 (e.g., a first sheath), the repositioning sheath 32 (e.g., a second sheath), and a closure device 70 at a vascular access site 72, among other suitable components and/or features, with the closure device 70 distal of the introducer sheath 30 and the introducer sheath 30 distal of the repositioning sheath 32.

The closure device 70 may be any suitable device configured to close the vascular access site 72 around the repositioning sheath 32 after the introducer sheath 30 has been removed from the vascular access site 72 and/or around the introducer sheath. The closure device 70 may be, but is not limited to, a suture, a clip, a clasp, adhesive, a bandage, and/or other suitable closure device. In one example and as depicted in FIG. 4, the closure device 70 may be a suture, where the suture is to be inserted through skin of the patient proximate the vascular access site 72 such that after insertion of the blood pump through the vascular access site 72, insertion of the repositioning sheath 32 into the vascular access site 72, and withdrawal of the introducer sheath 30 from the vascular access site 72, the suture may be tied around the repositioning sheath 32 to close the vascular access 72 around the repositioning sheath 32 and prevent blood from leaking out of the vascular access site 72. Alternatively or additionally, the closure device 70 may be used to close the vascular access site 72 with the introducer sheath 30 inserted therein, to close the vascular access site 72 about the elongate shaft 12, to close the vascular access site 72 without any medical device extending therethrough, and/or to close the vascular access site 72 in one or more other suitable situations.

When the closure device 70 is or includes a suture, any suitable suture may be utilized. Example suitable sutures include, but are not limited to, dissolvable sutures, non-dissolvable sutures, polymer sutures, polypropylene sutures, Perclose sutures, and/or other suitable sutures. In one example, one or more non-dissolvable polypropylene sutures, such as a Perclose suture, may be utilized as the closure device 70, but other suitable closure devices 70 are contemplated.

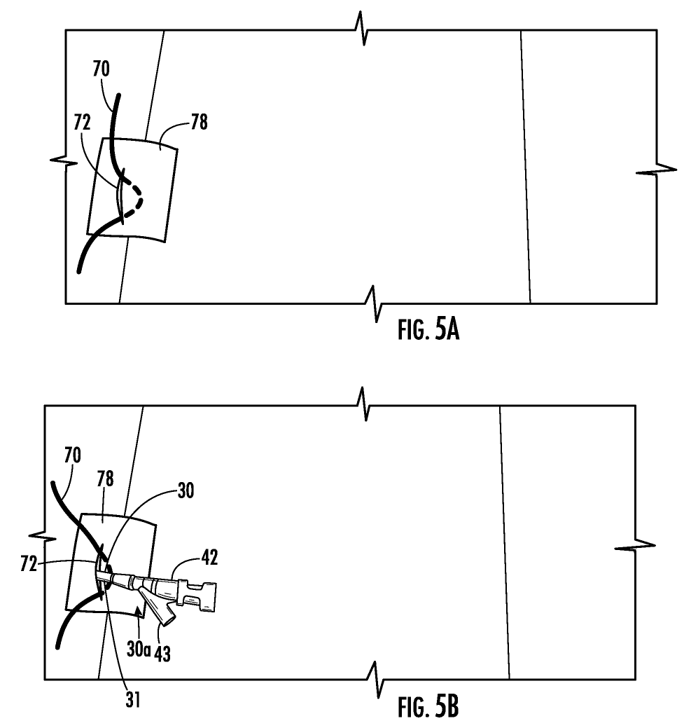

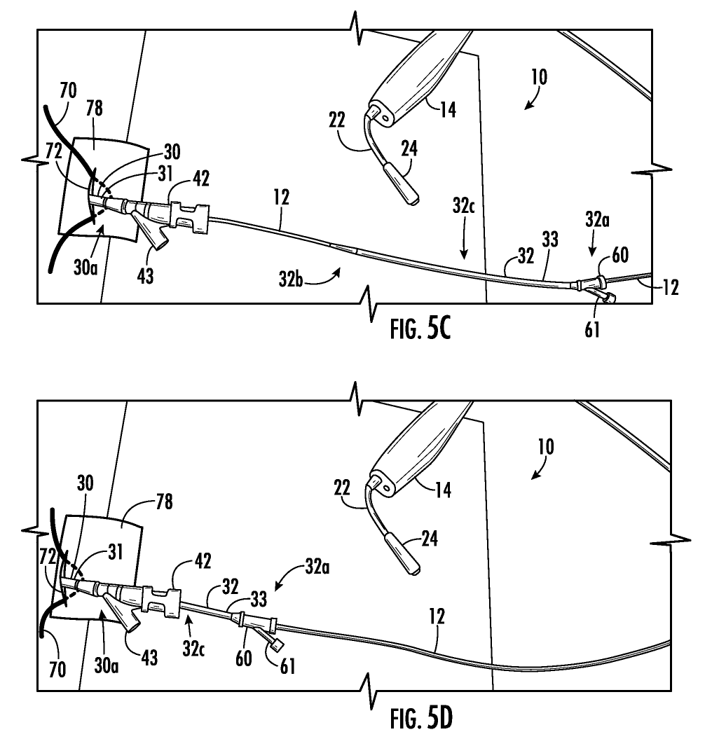

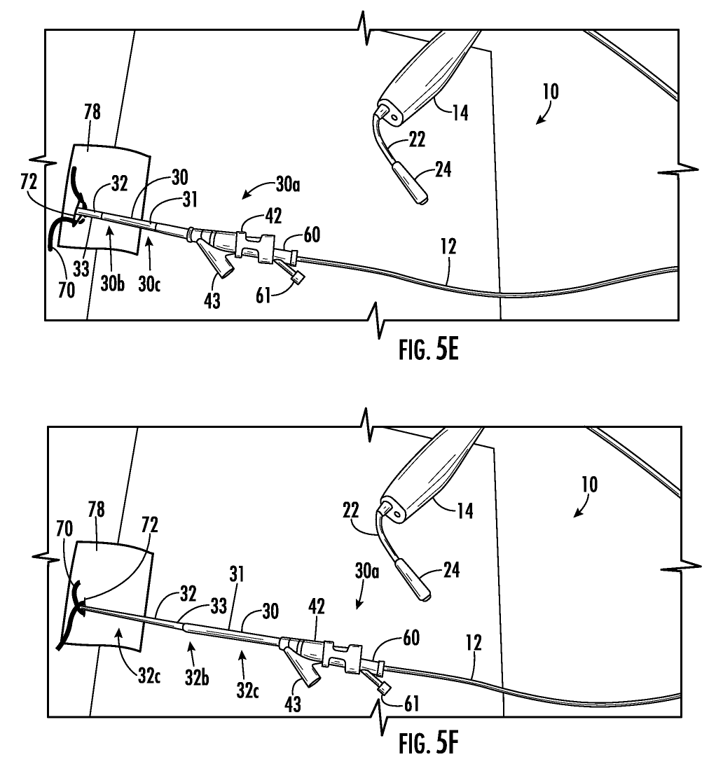

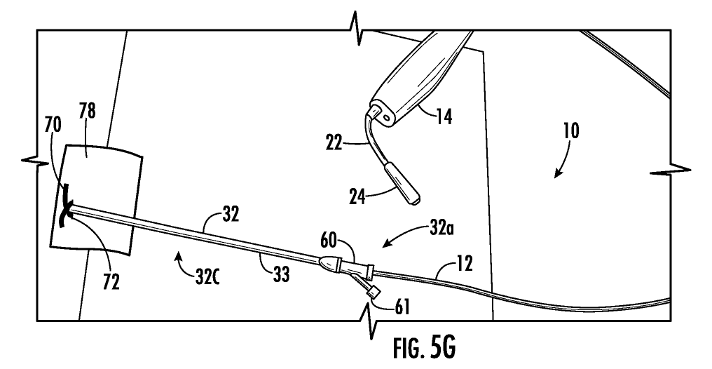

FIGS. 5A-5G schematically depict an illustrative technique for using the MCS system 10 that includes replacing the introducer sheath 30 (e.g., a first sheath) with the repositioning sheath 32 (e.g., a second sheath) at a vascular access site 72 in a subject 78. Although the illustrative technique for replacing the introducer sheath 30 with the repositioning sheath 32 is discussed with respect to the components of the MCS system 10, other suitable components may be utilized as desired. In some examples, the repositioning sheath 32 may be preloaded onto the elongate shaft 12 between the housing 14 and the blood pump 50. Although not required, the preloading of repositioning sheath 32 onto the elongate shaft 12 may be performed during the manufacturing and/or assembly of the MCS system 10.

FIG. 5A schematically depicts positioning the closure device 70 at, proximate to, and/or around the vascular access site 72 of the subject 78. In some examples, a vascular access site 72 may be created, in any suitable manner, providing access through skin of the subject 78 to a femoral artery or other suitable vessel of the subject 78. Then, the closure device 70 may be positioned at the vascular access site 72. Alternatively, the closure device 70 may be positioned at the subject 78 and the vascular access site 72 may be formed after positioning the closure device 70. In one example, when the closure device 70 is a suture, the closure device 70 may be inserted (e.g., threaded) into and/or through the skin of the subject 78 at or proximate the vascular access site 72 such that the suture may be tied at a later time to close the vascular access site around one or more components of the MCS system 10.

FIG. 5B schematically depicts the introducer sheath 30 of the MCS system 10 being inserted and/or advanced into the vascular access site 72 and into a vessel (e.g., the femoral artery and/or other suitable vessel) of the subject 78. When the introducer sheath 30 is inserted into the vascular access site 72, the hub 42 of the introducer sheath 30 may remain proximate to and proximal of the vascular access site 72. Although FIG. 5B depicts a configuration of the introducer sheath 30 with the port 43, the port 43 may be omitted and/or one or more additional or alternative ports may be utilized. Although not shown in FIG. 5B, a guidewire, a dilator, and/or other device may be utilized to facilitate inserting the introducer sheath 30 into the vascular access site 72 and/or the vessel.

FIG. 5C depicts the blood pump 50 (not shown in FIG. 5C) and the elongate shaft 12 of the MCS system 10 being inserted or advanced into and through the introducer sheath 30 and the vascular access site 72, with the elongate shaft 12 extending proximal from the hub 42 of the introducer sheath 30, through the repositioning sheath 32 to the housing 14. During advancing (e.g., insertion) of the blood pump 50 and the elongate shaft 12 through the introducer sheath 30 and into the subject 78, the repositioning sheath 32 may remain proximal of the introducer sheath 30, but other suitable configurations are contemplated in which the repositioning sheath 32 may be advanced into and/or through the introducer sheath 30 as part of advancing the blood pump to the target site.

FIG. 5D schematically depicts the introducer sheath 30 positioned in the vascular access site 72, with the repositioning sheath 32 being advanced distally along the elongate shaft 12, into the introducer sheath 30, and into the vascular access site 72. In some examples, the repositioning sheath 32 may be advanced in a distal direction over and/or with the elongate shaft 12, through a distal end of the introducer sheath 30, and to a location distal of the distal end (e.g., distal of the distal end region 30b) of the introducer sheath 30. Advancing the repositioning sheath 32 into the introducer sheath 30 and/or the subject 78 may occur with the introducer sheath 30 in the vascular access site 72, but this is not required and the introducer sheath 30 may be removed from the vascular access site 72 prior to, during, or after removing the introducer sheath 30 from the vascular access site 72.

If the repositioning sheath 32 includes an elongate shaft lock or other components configured to releasably fix the repositioning sheath 32 relative to the elongate shaft 12, the elongate shaft lock or other component may be adjusted from a locked position to an unlocked position to disengage the elongate shaft lock from the elongate shaft 12 prior to advancing the repositioning sheath 32 along the elongate shaft 12. Once relative movement of the elongate shaft 12 and the repositioning sheath 32 is possible, a user may advance the distal end of the repositioning sheath 32 into the introducer sheath 30 to a location across the vascular access site 72 and distal of the distal end of the introducer sheath 30, and into the vessel of the subject 78, but the repositioning sheath 32 may be advanced distally in one or more other manners. Alternatively or additionally, the repositioning sheath 32 may be advanced into the introducer sheath 30 with the elongate shaft 12 such that there is no relative movement between the elongate shaft 12 and the repositioning sheath 32 and, optionally, the elongate shaft lock may remain in the locked position.

FIG. 5E schematically depicts the introducer sheath 30 advancing proximally from the vascular access site 72 (e.g., fully withdrawn from the vascular access site 72 with the distal end of the introducer sheath 30 proximal of the vascular access site 72) to remove the introducer sheath 30 from the vascular access site 72 and the repositioning sheath 32 extending into vascular access site 72 (e.g., with the distal end of the repositioning sheath 32 distal of the distal end of the introducer sheath 30). Prior to removing the introducer sheath 30 from the vascular access site 72, the repositioning sheath 32 may be positioned within the vessel of the subject 78. In some examples, the repositioning sheath 32 and/or the introducer sheath 30 may have one or more markings that indicate when a distal tip of the repositioning sheath 32 is aligned with or distal of a distal tip of the introducer sheath 30 to indicate the repositioning sheath 32 is in the vessel of the subject 78 prior to withdrawing the introducer sheath 30 from the vascular access site 72. When advancing the introducer sheath 30 proximally, the hub 42 may advance over the repositioning sheath 32 to a location at or proximate to the hub 60 of the repositioning sheath 32 and, optionally, may releasably couple with the hub 60, and/or the hub 42 may be advanced to any other suitable location along the repositioning sheath 32.

FIG. 5F schematically depicts the introducer sheath 30 fully withdrawn from the vascular access site 72 and the closure device 70 closing the vascular access site 72 around the repositioning sheath 32. In some examples, the repositioning sheath 32 may remain within the subject 78 (e.g., with the vascular access site 72 and/or the vessel of the subject 78) while the blood pump 50 is within the subject 78 (e.g., for an extended dwell time of 1 hour, 2 hours, 6 hours, 12 hours, 1 day, 5 days, and/or other suitable dwell times). Although the elongate tube 33 of the repositioning sheath 32 is depicted in FIG. 5F as being much longer than the introducer sheath 30, it is contemplated that when the introducer sheath 30 is separable or otherwise removable, the introducer sheath 30 may have a length that is similar to, but less than the length of the repositioning sheath 32.

As depicted in FIG. 5F, the closure device 70 may be a suture and closing the vascular access site around the repositioning sheath 32 with the closure device 70 may include cinching and/or tying the suture around the repositioning sheath at the vascular access site 72. Although FIG. 5F depicts the closure device 70 closing the vascular access site 72 around the repositioning sheath 32 with the introducer sheath 30 removed from and proximal of the vascular access site, it is contemplated that the closure device 70 may close the vascular access site 72 around the introducer sheath 30 when the introducer sheath 30 is extending through the vascular access site 72 with or without the repositioning sheath 32 extending therethrough.

FIG. 5G schematically depicts the MCS system 10 with the introducer sheath 30 (not depicted in FIG. 5G) removed or separated from a location about the repositioning sheath 32 and/or the elongate shaft 12, with the repositioning sheath 32 advanced into the vascular access site 72. The tapering-up at the distal end of the repositioning sheath 32 may be utilized to provide a desired hemostasis control at the vascular access site 72 by adjusting an axial position of the repositioning sheath 32 relative to the vascular access site 72. In some examples, the introducer sheath 30 may be removed from a location about the repositioning sheath 32 while the repositioning sheath 32 is inserted into the vascular access site 72, but other suitable configurations are contemplated. Although the introducer sheath 30 is depicted in FIG. 5G as being removed after closing the vascular access site 72 with the closure device 70, the introducer sheath 30 may be removed from a location about the repositioning sheath 32 at one or more other suitable times before, during, or after closing the vascular access site 72.

The introducer sheath 30 may be removed from around the repositioning sheath 32 in any suitable manner. In some examples, the introducer sheath 30 may be a separable sheath that may have two or more portions that are longitudinally separable from one another. For example, when the introducer sheath 30 has two or more longitudinally separable components, the components may be separated from one another and removed from the MCS system 10. In one example, the introducer sheath 30 may include one or more perforation lines and/or pull lines between two separable components to facilitate separating the components of the introducer sheath 30. The longitudinally separable components of the introducer sheath 30 may or may not include the hub 42.

Although not depicted, the MCS system 10 may include one or more suture pads. When included, the suture pads may be fixed to the subject 78 proximate the vascular access site 72 to facilitate maintaining a position of the repositioning sheath 32 and/or the elongate shaft 12 relative to the vascular access site 72 to mitigate movement that may affect a positioning of the blood pump 50 at a target site.

Further, if the hub 60 includes an elongate shaft lock, the elongate shaft lock may be adjusted from an unlocked position or configuration to a locked position or configuration to secure the repositioning sheath 32 to or relative to the elongate shaft 12. The fixing of the repositioning sheath 32 relative to the elongate shaft 12 may occur at any suitable time after positioning the repositioning sheath 32 through the vascular access site 72 and into the vessel of the subject 78.

The schematic methods and techniques discussed herein may be used together, unless expressly indicated otherwise. Further, the order of implementation of the steps discussed herein may be in other suitable orders, unless expressly indicated otherwise, may include one or more intervening steps discussed herein or otherwise.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical device system, the system comprising:
a medical device;
an elongate shaft coupled with the medical device and extending proximally from the medical device;
a first sheath having a first hub and a first elongate tube, wherein the first elongate tube extends in a distal direction from the first hub, and a sheath length extending from a proximal end of the first hub to a distal end of the first elongate tube, the first elongate tube has a lumen with an inner diameter and the inner diameter is expandable from a first inner diameter to a second inner diameter; and
a second sheath having a second elongate tube, the second elongate tube having a tube length that is longer than the sheath length, and
wherein the first sheath and the second sheath are configured to longitudinally advance over the elongate shaft and a distal end of the second sheath is configured to advance through the lumen and extend distally beyond the distal end of the first elongate tube.

2. The system of claim 1, wherein the second sheath comprises a second hub and the second elongate tube extends in the distal direction from a distal end of the second hub.

3. The system of claim 1, wherein the first sheath is an introducer sheath and the first elongate tube is configured to expand to the second inner diameter in response to receiving the medical device in the lumen.

4. The system of claim 1, wherein the first inner diameter is in a range of 2.33 millimeters (mm) to 3 mm and the second inner diameter is in a range of 4.67 mm to 6.67 mm.

5. The system of claim 1, wherein the second sheath is a single lumen sheath.

6. The system of claim 1, further comprising:
a closure device configured to extend around the second elongate tube and close a patient access site within which the second elongate tube is extending.

7. The system of claim 6, wherein the closure device is a suture.

8. The system of claim 1, wherein the first sheath is separable into two or more portions for removal from the elongate shaft after the blood pump has been inserted through the lumen.

9. The system of claim 1, further comprising:
a housing, and
wherein the elongate shaft extends between a proximal end of the medical device and a distal end of the housing, and
wherein the second sheath is located about the elongate shaft between the housing and the medical device.

10. The system of claim 9, wherein the medical device is a blood pump configured to pump blood from a ventricle of a heart of a patient to vasculature of the patient.

11. A method of using a medical device system, the method comprising:
positioning a closure device at a vascular access site of a patient;
advancing a medical device and an elongate shaft extending proximally from the medical device through a first sheath extending into the vascular access site;
advancing a distal end of a second sheath over the elongate shaft and through a distal end of the first sheath to a location distal of the distal end of the first sheath; and
closing the vascular access site around the second sheath using the closure device.

12. The method of claim 11, wherein the closure device is a suture, positioning the closure device at the vascular access site includes threading the suture through skin of the patient, and closing the vascular access site around the second sheath includes tying the suture around the second sheath to close the vascular access site around the second sheath.

13. The method of claim 11, wherein the second sheath includes a taper and advancing the distal end of the second sheath to a location distal of the distal end of the first sheath includes positioning the taper to control hemostasis at the vascular access site.

14. The method of claim 11, further comprising:
advancing the first sheath through the vascular access site after positioning the closure device at the vascular access site.

15. The method of claim 11, further comprising:
removing the first sheath from the vascular access site; and
removing the first sheath from the second sheath and the elongate shaft.

16. The method of claim 11, wherein the first sheath includes an elongate tube with an inner diameter configured to expand from a first inner diameter to a second inner diameter as the medical device is advanced through the first sheath.

17. A medical device system, the system comprising:
a first sheath having a first hub and a first elongate tube extending in a distal direction from the first hub, the first elongate tube has a lumen with an inner diameter;
a medical device;
a housing;
an elongate shaft coupled with the medical device and extending proximally from the medical device to the housing;
a second sheath having a second elongate tube positioned about the elongate shaft, the second elongate tube has an outer diameter that is less than the inner diameter of the first elongate tube; and
a closure device configured to close a vascular access site around the second sheath.

18. The system of claim 17, wherein the second sheath is configured to adjust between a first position entirely proximal of the first sheath and a second position in which the second elongate tube extends distal of a distal end of the first sheath.

19. The system of claim 17, wherein the first elongate tube is configured to expand from a first inner diameter to a second inner diameter.

20. The system of claim 17, wherein the closure device is a suture.
